(19) United States
(12) Patent Application Publication
(10) Pub. No.: US 2005/0136015 A1
(43) Pub. Date: Jun. 23, 2005

(54) Topical use of halosalicylic acid derivatives

(76) Inventors: Derrick B. McKie, Brooklyn, NY (US); Gopinathan K. Menon, Wayne, NJ (US); Ying Ye, Martinsville, NJ (US)

Correspondence Address: Anthony M. Santini;Avon Products, Inc., Avon Place, Suffern, NY, 10901, US

(21) Appl. No.: 10/738,411

(22) Filed: Dec. 17, 2003

(51) Int. Cl. A61K 7/06; A61K 31/60

(52) U.S. Cl. 424/70.1; 514/159

(57) ABSTRACT

Halosalicylic acid compounds of the present invention, having at least one halogen substituent on the aromatic ring, are employed to produce desquamation of skin, treat nail disorders or dandruff, remove calluses, control acne and excess sebum production, reduce skin pore size or control blackheads. Topical compositions containing the halosalicylic acid compound and a cosmetically acceptable vehicle are disclosed.

### BACKGROUND OF THE INVENTION

1. Field of the Invention

The present invention provides a method for treating nail disorders, dandruff, callus, accelerated sebum production, enlarged pores, blackheads, acne or skin requiring desquamation by applying to an affected area an effective amount of a halosalicylic acid derivative of the invention. Cosmetic topical compositions containing halosalicylic acid derivatives and useful in such method are also provided.

2. Description of the Related Art

Halosalicylic acid compounds are known in the art.

U.S. Pat. No. 5,817,666 discloses the use of about 0.1 to 10% 5-fluorouracil and about 5% to 70% of halogenated carboxylic acids, keto acids, salicylic acid, and combinations thereof as a superficial dermal peel in the treatment of actinic skin damage. Patentees indicate that the acids can be present in the free form or as a salt.

U.S. Pat. No. 5,558,871 discloses a method of treating acne or ageing (wrinkles, fine lines and complexion) by applying to the skin a salicylic acid derivative of formula I. Patentees indicate that their composition can be used to treat the body and face, including the scalp and nails. The composition contains a salicylic acid derivative having a keto substituent (R—CO—) at the 5^{th} ring position. A vegetable oil is employed to solubilize the salicylic acid derivative.

U.S. Pat. No. 5,667,789 discloses the use of a salt of a salicylic acid derivative of depicted general formula I as a stabilizer for an oil-in-water emulsion. Salicylic acid derivatives of the formula I of the '789 patent are substituted at the 5^{th} ring position by the group R. R is defined as “a saturated, linear, branched, or cyclic aliphatic, alkoxy, alkanoyloxy, alkanoyl, or alkyl carboxy group, each group having 2 to 22 carbon atoms and each group optionally substituted with a least one substitiuent selected from the group consisting of halogen, trifluoromethyl . . . ; or an unsaturated, linear, branched, or cyclic alkenyl, alkenyloxy, alkenoyloxy, alkenoyl or alkenyl carboxy group having one or more conjugated or non-conjugated double bonds, each group having 2 to 22 carbon atoms ach each group optionally substituted with at least one substituent selected from the group consisting of halogen, trifluoromethyl, . . . ” Clearly, the disclosure of substitution at the 5^{th} ring position by groups that contain from 2 to 22 carbon atoms and that can contain halogen or trifluoromethyl groups substituted thereon is not a disclosure of a halogen group, methyl group (C₁) or trifluoromethyl substituted at the 5^{th} ring position.

U.S. Pat. No. 6,281,203 discloses compositions for treating acne or aging of the skin. The compositions contain (i) salicylic acid and/or at least one salicylic acid derivative, (ii) at least one ester of a fatty acid and glucose and/or alkyl glucose, and (iii) at least one oxyethylenated ether of a fatty acid ester of glucose and/or alkyl glucose. Suitable salicylic acid derivatives include those of the general formula I disclosed therein, or a salt thereof. Patentees appreciate that the 5^{th} position on the ring can be substituted by a saturated, linear, branched or cyclized aliphatic hydrocarbon group, among others. Patentees state that these groups may contain from 1 to about 22 carbon atoms and may be substituted with at least one substituent chosen from halogen atoms, the trifluoromethyl group, hydroxyl groups . . . etc. Patentees specifically mention 5-methylsalicylic acid (R₁ being methyl). Although patentees state that R₁ can be C₁-C₂₂ alkyl and that the alkyl group can be substituted with at least one substituent chosen from a group that includes halogen, patentees fail to specifically disclose the 5-trifluoromethyl derivative.

U.S. Pat. No. 5,723,109 discloses salicylic acid derivatives for topical application to the skin of the face and/or body, to lighten the skin or treat pigmented blemishes without desquamation or peeling of the skin. The salicylic acid derivatives are substituted at the 5^{th} ring position with the keto group R—CO—, wherein R is a linear, branched or cyclic saturated aliphatic group or an unsaturated group containing one or a number of double bonds, which may or may not be conjugated, these groups containing 2 to 22 carbon atoms and being able to be substituted by at least one substituent from a group that includes, among others, halogen atoms and trifluoromethyl. The halosalicyclic acid derivatives of the present invention lack the 5-keto substituent present in the salicylic acid derivatives of the '109 patent. Moreover, this patent speaks to preventing desquamation, which is contrary to the present invention.

Rhee et al, (1989) Yakhak Hoeji, Vol. 33, No. 2, p. 87-100 “Quantum Chemical Analysis of Structure-Activity Relationships in Salicylic Acids as Anti-inflammatory Drugs” evaluated 5-bromosalicylic acid, 5-chlorosalicylic acid, 3,5-dichlorosalicylic acid, 5-chlorosalicylic acid methyl ester, 3-fluorosalicylic acid, 4-fluorosalicylic acid, 5-fluorosalicylic acid, 6-fluorosalicylic acid, 3-fluoro-5-phenylsalicylic acid, 5-(2-fluorophenyl)salicylic acid, 5-(3-fluorophenyl)salicylic acid, 5-(4-fluorophenyl)salicylic acid, 5-(4-chlorophenyl)salicylic acid, 5-(2,4-difluorophenyl)salicylic acid, 3-methyl-5-(4-fluorophenyl)salicylic acid, and 5-(2-methyl-4-fluorophenyl)salicylic acid, among others, for structure-activity relationship with respect to anti-inflammatory potency. The study appears to be directed to systemic activity, rather than topical activity. Thus, it would not lead one skilled in the art to topically use any of the salicylic acid derivatives disclosed therein.

5-chlorosalicylic acid was tested and found to be non-mutagenic (see “Mutagenic activity of 2-chloro-4-nitroaniline and 5-chlorosalicylic acid in Salmonella typhimarium: Two possible metabolites of niclosamide”, Inst. Invest. Biomed, Univ. Nac. Auton. Mexico, Mexico City, 04510 Mex.).

5-bromosalicylic acid and 5-chlorosalicylic acid have been applied preharvest to reduce sugars and color in processed potatoes (see Proc. Plant Growth Regul, Soc. AM. (1990) 17^{th}, 88-93).

Thus, the prior art has failed to appreciate the topical use of salicylic acid derivatives having at least one halogen substituent substituted directly on the aromatic ring.

### SUMMARY OF THE INVENTION

The present invention relates to dermatological and cosmetic compositions containing a salicylic acid derivative and to the use of such compositions for desquamation of the skin, for accelerated sebum and acne control, for treatment of nail disorders, for treatment of dandruff, for callus removal and/or for reduction of skin pore size and control of blackheads.

### DETAILED DESCRIPTION OF THE INVENTION

The halo salicylic acid derivatives of the present invention conform to the following general formula I:

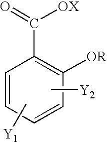

wherein X is hydrogen, a C₁-C₈ alkyl group, preferably methyl, a C₂-C₈ alkenyl group, or a cosmetically acceptable cation;
R is hydrogen, C₁-C₁₈ alkyl or C₁-C₁₈ alkyl substituted with at least one Cl, Br, F or I group; and
Y₁ and Y₂ are, independently, hydrogen, Cl, Br, F, I, methyl substituted with one to three Cl, Br, F or I groups, phenyl, or phenyl substituted with at least one substituent selected from the group consisting of C₁-C₁₈ alkyl, Cl, Br, F and I, with the proviso that at least one of Y, and Y₂ is Cl, Br, F or I.

The preferred haloalkyl group is trifluoromethyl.

Preferred compounds of formula I included 5-bromosalicylic acid, 5-chlorosalicylic acid, 5-fluorosalicylic acid, 5-iodosalicylic acid, 3-fluorosalicylic acid, 4-fluorosalicylic acid, 6-fluorosalicylic acid, 5-chlorosalicylic methyl ester, 3-methyl-5-(4-fluorophenyl)salicylic acid, 5-(2,4-difluorophenyl)salicylic acid, 5-(3-fluorophenyl)salicylic acid, 5-(2-fluorophenyl)salicylic acid, 5-(4-fluorophenyl)salicylic acid, 5-(2-methyl-4-fluorophenyl)salicylic acid, 6-fluorophenylsalicylic acid, 3-fluoro-5-phenylsalicylic acid, and 5-trifluoromethylsalicylic acid.

5-chlorosalicylic acid, 5-fluorosalicylic acid, 5-bromosalicylic acid, 5 iodosalicylic acid and 5-trifluoromethylsalicylic acid are more preferred.

5-chlorosalicylic acid is most preferred.

When the composition is to be employed as a foot cream or lotion for removal of calluses, compounds of the formula I, wherein at least one of Y₁ and Y₂ is methyl substituted with one to three Cl, Br, F or I groups, are preferred. Compounds of formula I wherein at least one of Y₁ and Y₂ is trichloromethyl are particularly preferred for callus removal as the trichloromethyl group enhances the lipophilic nature of compounds of formula I making skin penetration more facile.

Compositions in accordance with the present invention comprise (i) an amount of a halosalicylic acid derivative of formula I effective for desquamation of the skin, accelerated sebum and/or acne control, treatment of nail disorders, treatment of dandruff, callus removal, reduction of skin pore size or control of blackheads, and (ii) a cosmetically acceptable vehicle for the halosalicylic acid derivative of formula I.

The halosalicylic acid derivative of formula I is generally present in an amount of about 0.001% to about 10%, preferably, about 0.01% to about 5%, more preferably, about 0.1% to about 2.5%, even more preferably, about 0.25% to about 2.2%, and most preferably, about 0.5% to about 2.0%, by weight based on the total weight of the composition.

The antimicrobial activity of 5-chlorosalicylic acid, a representative compound of formula I, was compared to that of salicylic acid. Salicylic acid's minimal lethal concentration was determined. The results are set forth in Table 1, which follows:

As is evident from the data of Table 1, salicylic acid is bacteriocidal against all of the test microorganisms.

TABLE 1
MLC Test Results for Salicylic Acid
% Concentration for Bacterioidal Activity

| Test Microorganism | Salicylic Acid |
|---|---|
| Pseudomonas aeruginosa | 0.0625 |
| Escherichia coli | 0.125 |
| Staphylococcus epidermidis | 0.125 |
| Candida albicans | 0.25 |
| Aspergillus niger | 0.25 |

Because of solubility issues with 5-chlorosalicylic acid and the difficulty of growing Propionibacterium acnes, a Zone of Inhibition Test based on the National Center of Clinical Laboratories Standards protocol, was used to compare the activity of 5-chlorosalicylic acid to that of salicylic acid. Isopropyl palmitate was employed as the solvent for the salicylic acid and 5-chlorosalicylic acid.

It should be appreciated that the Zone of Inhibition Test does not differentiate between bacteriocidal and bacteriostatic antimicrobial activity.

The zone of inhibition test results are set forth-in Tables 2 through 4, which follow.

The results of Tables 2 through 4 demonstrate that 5-chlorosalicylic acid is more active against Propionibacterium acnes than salicylic acid. The combination of 1.66 wt. % salicylic acid and 1 wt. % 5-chlorosalicylic acid gave the best results. However, when combined with the 5-chlorosalicylic acid concentrations of the present invention, from about 0.5 wt. % to about 2 wt. % salicylic acid may be used.

TABLE 2
Zone of Inhibition Test Results for Salicylic Acid
and 5-Chlorosalicylic Acid

| | Diameter of Zone of Inhibition (Millimeters) | | | |
|---|---|---|---|---|
| | | | 5-Chlorosalicylic | |
| | Salicylic Acid-1000 μl | | Acid-1000 μl | |
| Test Microorganism | 1.66% | 0.83% | 1.0% | 0.5% |
| Propionibacterium acnes | 15.39 | 0 | 17.62 | 4.87 |
| Staphylococcus epidermidis | 13.62 | 4.5 | 13.15 | 3.59 |
| Staphylococcus aureus | 6.38 | 0 | 3.04 | 0 |
| Escherichia coli | 3.67 | 0 | 0 | 0 |
| Pseudomonas aeruginosa | 0 | 0 | 0 | 0 |
| Candida albicans | 11.16 | 4.6 | 2.96 | 0 |
| Aspergillus niger | 6.72 | 0 | 3.38 | 0 |

TABLE 3
Zone of Inhibition Test Results for Mixtures of Salicylic Acid and
5-Chlorosalicylic Acid - 1000 μl

| | Diameter of Zone of Inhibition (Millimeters) | | |
|---|---|---|---|
| | 1.66% Salicylic Acid + 1% | 1.66% Salicylic Acid + 0.5% | 1.66% Salicylic Acid + 0.16% |
| | 5-Chlorosalicylic | 5-Chlorosalicylic | 5-Chlorosalicylic |
| Test Microorganism | Acid | Acid | Acid |
| Propionibacterium acnes | 22.59 | 23.02 | 14.32 |
| Staphylococcus | 20.82 | 18.92 | 17.42 |
| epidermidis | | | |
| Staphylococcus aureus | 12.66 | 10.43 | 9.91 |
| Escherichia coli | 7.61 | 6.76 | 5.83 |
| Pseudomonas aeruginosa | 0 | 0 | 0 |
| Candida albicans | 12.96 | 12.98 | 12.26 |
| Aspergillus niger | 11.18 | 8.20 | 7.92 |

TABLE 4
Zone of Inhibition Test Results for Mixtures of Salicylic Acid and
5-Chlorosalicylic Acid - 1000 μl

| | Diameter of Zone of Inhibition (Millimeters) | | |
|---|---|---|---|
| | 0.83% Salicylic Acid + 1% | 0.83% Salicylic Acid + 0.5% | 0.83% Salicylic Acid + 0.16% |
| | 5-Chlorosalicylic | 5-Chlorosalicylic | 5-Chlorosalicylic |
| Test Microorganism | Acid | Acid | Acid |
| Propionibacterium acnes | 22.61 | 20.53 | 8.96 |
| Staphylococcus | 16.73 | 12.61 | 12.12 |
| epidermidis | | | |
| Staphylococcus aureus | 9.79 | 6.72 | 5.23 |
| Escherichia coli | 5.77 | 5.02 | 0 |
| Pseudomonas aeruginosa | 0 | 0 | 0 |
| Candida albicans | 11.27 | 9.15 | 7.99 |
| Aspergillus niger | 7.74 | 5.11 | 0 |

The exfoliation (desquamation) activity of compounds of formula I, as represented by 5-chlorosalicylic acid, was compared to that of salicylic acid. D-SQUAME skin surface sampling Discs (CuDerm Corporation) were employed. The disc was applied to a clean, dry skin surface and pressed firmly for a few seconds using the thumb or fingertips. The disc was then transferred to a black square on the storage card. The disc was viewed at an angle with strong light and compared with 5 reference patterns provided by CuDerm Corporation. Very dry skin produces a heavy amount of scaling similar to pattern 5. Normal skin produces a few areas of small clumps of cells or a fine even single layer of cells.

The scoring scale employed was as follows:

| | |
|---|---|
| 0 | No evidence of any cells. |
| ± | (Barely Preceptible)—few scattered single, fine cells throughout |
| | D-SQUAME site. |
| 1 | (Minimal)—minimal scattering of single, fine cells unevenly |
| | distributed throughout the D-SQUAME site. |
| 2 | (Mild)—moderate scattering of single and/or clustered poor quality, |
| | (large/distorted) cells throughout the D-SQUAME site; cell mass is |
| | slightly dense in some, but not all, of the D-SQUAME site. |
| 3 | (Moderate)—moderate to heavy scattering of clustered, poor quality |
| | large/distorted cells throughout the D-SQUAME site; cell mass is |
| | moderately dense. |
| 4 | (Moderate/Heavy)—thick, dense cell mass throughout the entire |
| | D-SQUAME site. |
| 5 | (Heavy)—thick, extremely dense cell mass of “sheets” of stratum |
| | corneum throughout entire D-SQUAME site. |

A mixture of 0.5% chlorosalicylic acid and 0.5% salicylic acid was also tested. The vehicle was tested, as a control. The vehicle (ANEW All-In-One SPF-15 Self-Adjusting Perfecting Creme base without the glycolic acid) employed was the same in all cases, only the test compound(s) differed.

The results are set forth in Table 5 below, wherein CLSA stands for 5-chlorosalicylic acid and SA stands for salicylic acid.

The skin irritation (PII) of each test formulation was determined and is also set forth in Table 5.

TABLE 5

Active Ingredient

| CLSA | SA | PII | D-SQUAME Score |
|---|---|---|---|
| 0.5% | 0 | 0.00 | 2.44 |
| 0.5% | 0.5% | 0.00 | 2.67 |
| 0 | 0.5% | 0.00 | 1.97 |
| 0 | 1% | 0.00 | 2.28 |
| 0 | 2.% | 0.00 | 2.56 |
| 0 | 0 | 0.00 | 1.94 |

It should be appreciated that in considering the results of Table 5, a D-SQUAME score of, for example, 2.44, means that the criteria for number grade 2 has been met and has in fact been exceeded. 2.44 in essence represents an in-between grade. Thus, a D-SQUAME score of 1.97 meets the criteria for grade 1 and comes very close to meeting the criteria for grade 2.

As is evident from the results set forth in Table 5, no significant irritation was observed with any of the tested formulation. All were acceptably mild.

The results of Table 5 show that:
0.5% chlorosalicylic acid was:
   equivalent in exfoliation activity to the combination of 0.5% chlorosalicylic acid/0.5% salicylic acid;
   significantly more exfoliating than 0.5 salicylic acid;
   equivalent in exfoliation activity to 1.0% salicylic acid;
   equivalent in exfoliation activity to 2.0% salicylic acid; and
   significantly more exfoliating than the base vehicle (containing no CLSA or SA).
0.5% chlorosalicylic acid/0.5 salicylic acid was:
   significantly more exfoliating than 0.5% salicylic acid;
   equivalent in exfoliation activity to 1.0% salicylic acid;
   equivalent in exfoliation activity to 2.0% salicylic acid; and
   significantly more exfoliating than the base vehicle (containing no CLSA or SA).
Surprisingly, chlorosalicylic acid at 0.5%, either alone or in combination with 0.5% salicylic acid, provides exfoliation activity comparable to that of 1.0% and 2.0% salicylic acid.

Due to their partition and diffusion coefficients, halosalicylic acid derivatives of formula I will rapidly penetrate skin. This has been confirmed with calculations for 5-chlorosalicylic acid from the skin penetration model (“Modelling dermal exposure and absorption through the skin”, W. F. ten Berge, DSM, Heerlen, the Netherlands, http://home.planet.nl/~wtberg/skinperm.html).

The calculated parameters for 5-chlorosalicylic acid and salicylic acid are set forth in Table 6, which follows:

TABLE 6

| | 5-Chlorosalicylic | Salicylic |
|---|---|---|
| Skin Penetration Parameter | acid | acid |
| Skin permeability [cm/hour] × 103 | 30.2 | 6.3 |
| Storage in stratum corneum in hour Msc | 447 | 40.24 |
| [g/cm²] | | |
| Total uptake by skin in 1 hour Muptake | 579 | 71.52 |
| [g/cm²] | | |
| Time for uptake from formulation with | 1.93 | 4.2 |
| 0.5% of hydroxy acid t [min] | | |

As noted heretofore, the halosalicylic acid derivatives of formula I can be employed to treat enlarged skin pores and blackheads. The halosalicylic acid derivatives of formula I lyse follicular plugs and, because of their greater permeation through skin (as compared to salicylic acid), they produce excellent plug resolution.

When the halosalicylic acid derivative of formula I is employed for reduction of skin pore size, it is preferably employed in a composition containing one or more co-actives that target multiple steps leading to enlarged skin pores. Such co-actives include:
(i) one or more RAR/RXR agonists, such as phytol, which act to prevent hyperkeratinization in the follicular infundibular and also to clear the pore passage.
(ii) one or more 5-alpha-reductase inhibitors, such as oleanolic acid, which act to reduce sebum production (leading to less pore plug build up) and reduce the need for a larger pore passage.

Compositions containing a halosalicylic acid derivative of formula I, intended for use in the treatment of enlarged pores, may contain:
(i) a halosalicylic acid derivative of formula I, in an amount of about 0.01% to about 10%, preferably, about 0.1% to about 2.5%, more preferably, about 0.25% to about 2.2%, most preferably, about 0.5% to about 2.0%, by weight, based on the total weight of the composition;
(ii) an RAR/RXR agonist, in amount of about 0.0001% to about 50%, preferably, about 0.01% to about 20%, more preferably, about 0.1% to about 15%, most preferably, about 0.5% to about 5%, by weight, based on the total weight of the composition; and
(iii) a 5-alpha-reductase inhibitor, in an amount of about 0.01% to about 5%, preferably, about 0.1% to about 0.5%, by weight, based on the total weight of the composition.

Preferably, the composition also contains a mattifying agent, in other words, an agent that acts to minimize the color contrast between an enlarged pore and its surrounding skin thereby optically concealing the enlarged pore.

When a mattifying agent, i.e., an agent that reduces luster or shine, is employed in the composition of the invention it is generally present in amount of 0.01% to about 20%, preferably, about 0.1% to about 10%, more preferably, about 0.25% to about 5%, most preferably, about 0.5% to about 2.0%, by weight, based on the total weight of the composition.

RAR/RXR agonists that can be employed include, for example, phytol, isophytol, phytol derivatives, isophytol derivatives, retinoids, and mixtures thereof. Phytol and retinol are preferred.

“Phytol derivatives”, as used herein and in the claims that follow, connote those organic compounds that conform to the structural formula:

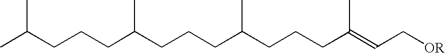

wherein R is selected from a group of substituents that includes hydrogen, as well as cyclic and acyclic hydrocarbon residues, which may contain one or several unsaturated bonds and/or heteroatomic substituents. The preferred substituents are hydrogen, acyls and cyclic or linear alkyls.

The term “phytol”, as used herein and in the claims that follow, includes phytol, isophytol, phytol derivatives, isophytol derivatives, phytol precursors, isophytol precursors, isophytol metabolites and phytol metabolites, preferably phytanic acid.

5-alpha-reductase inhibitors that can be employed include, for example, oleanolic acid, saw palmetto, finasteride, and mixtures thereof. Oleanolic acid is preferred.

Mattifying agents that can be employed include, for example, dimethicone blends, silica, and mixtures thereof. Dimethicone blends are preferred.

The compositions of the present invention can be formulated as ointments, creams and lotions (for example, oil-in-water or water-in-oil emulsion based), gels, mousses, suspensions, solutions, aerosols, sprays, sticks, patches or any other cosmetically and dermatological acceptable dosage form.

The compositions of the present invention can contain preservatives, germicides, antibacterial agents, vitamins agents, sunscreen agents, antioxidants, perfume agents, emollients, humectants, solvents, thickeners, bulking agents, fillers, ultraviolet light absorbers, skin cooling agents, penetration enhancers, gellents, waxes, clays, polymers, stabilizers, as well as other agents typically employed in cosmetic and dermatological products.

The compositions can also contain other actives provided they are compatible with the halosalicylic acid derivatives of formula I in that by their incorporation they do not prevent the benefits of the halosalicylic acid derivatives from being realized.

Actives that can be incorporated in the compositions of the present invention include, for example:
(i) antiaging actives, such as alpha hydroxy acids, beta hydroxy acids, and retinoids, (the term “retinoid” includes: (1) retinol; (2) esters of retinol with carboxylic acids of 1 to 24 carbon atoms, such as retinyl acetate, retinyl propionate, retinyl butyrate, retinyl octanoate, retinyl laurate, retinyl palmitate, retinyl oleate, retinyl linoleate; (3) esters of retinol having an alpha-hydroxy carboxylic acid; (4) ether derivatives of retinol, including alkyl ether, ethers derived from glycolic acid, as well as glycolate ester and amide, such as retinyl glycolyl ether; (5) retinaldehyde; (6) retinoic acid; (7) esters of retinoic acid with alcohols of 1 to 24 carbon atoms; (8) isotretinoin as well as synthetic retinoid mimics, and derivatives of the foregoing, as well as others that bind to RAR receptors; (9) cis- and trans-isomers of the foregoing retinoids; (10) salts of the foregoing retinoids; and (11) mixtures of the any of the foregoing compound);
(ii) anti-inflammatory agents, such as, salicylic acid, boswellic acid, curcumin, tetrahydrocurcumin, ferulic acid and its derivatives, rosmarinic acid, catechins, and bisabolol;
(iii) sunscreens, such as oxybenzone, octylsalicylate, octylmethoxycinnamnate, octocrylene, titanium dioxide, zinc oxide, butyl methoxydibenzoyl methane, methylene bis-benzotriazoyl tertramethyl butylpenol, bis-ethylhexyl oxyphenol methoxyphenol triazine;
(iv) antioxidant agents, such as, Vitamin C, Vitamin E, gallic acid and its derivatives, ferulic acid and its derivatives, nitrones, N-tertbutyl-nitrone, I-(4-pyridol-1-oxide)-N-tertbutyl-nitrone, curcumin, tetrahydrocurcumin, 6-hydroxy-2,5,7,tetramethylchroman-2-carboxylicacid, uric acid, reductic acid, tannic acid, rosmarinic acid, tocopherol and its derivatives, catechins, and mixtures thereof. Other suitable antioxidants are those that have one or more thiol functions (—SH), in either reduced or non-reduced form, such as glutathione, lipoic acid, thioglycolic acid, and other sulfhyryl compounds. The antioxidant may be inorganic, such as a sulfite, bisulfite, metabisulfite, or another inorganic salt and/or acid containing sulfur;
(v) collagen enhancing agents, such as, Vitamin C, ascorbyl-phosphoryl-cholesterol and clara extract (sophora augustifolia);
(vi) elastase inhibitors, such as, oleic acid, perinaric acid, honeysuckle extract (Lonicera caprifolium);
(vii) exfoliants, such as alpha-hydroxy acids, beta-hydroxy acids, keto acids, niacinamide, oxa acid, oxa diacid, (particularly trioxaundecanedioic acid) and mixtures thereof (alpha hydroxy acids, particularly, lactic acid and glycolic acid, are preferred); and
(viii) oil absorbing polymers, such as olefin block polymers.

It should be noted that when the composition of the present invention is intended for use in controlling excess sebum production, it may be desireable to include in the composition an oil absorbing material such as bentonite, rice starch, silica, calcium sulfate or mixtures thereof.

When the composition of the present invention is intended for use in treating dandruff, controlling acne, providing anti-ageing of skin (i.e., providing skin desquamation), treating inflammatory conditions of the skin or treating nail disorders, the composition of the present invention preferably employs as the halosalicylic acid compound of formula I, a chlorosalicylic acid compound, preferably in an amount of about 0.1% to about 10%, by weight, based on the total weight of the composition.

Chlorosalicylic acid compounds of formula I are highly lipophylic and due to their favorable partition and diffusion coefficients, as compared to salicylic acid, they are expected to rapidly penetrate skin. Calculations for 5-chlorosalicylic acid from the skin penetration model have confirmed this.

The following examples are offered to illustrate the present invention and are not intended to be limiting in any respect.

It should be noted that, unless indicated to the contrary, all percentages are percent by weight, based on the total weight of the composition.

### EXAMPLE 1

| Part | Ingredients | Wt. % |
|---|---|---|
| A | Glyceryl stearate | 10.0 |
| | Propylene glycol dicaprylate/dicaprate | 8.0 |
| | Cetearyl alcohol and sodium cetearyl sulfate | 5.0 |
| B | Propylene glycol | 3.0 |
| | Allantoin | 0.2 |
| | Methylparaben | 0.1 |
| | 5-Chlorosalicylic acid | 4.0 |
| | Sodium 5-chlorosalicylate | 1.7 |
| | Demineralized water | 67.7 |
| C | Fragrance | 0.3 |

The part A components are melted and paddle mixed together at 75°-80° C. The part B components are separately paddle mixed and brought to the same temperature as part A. Part A is milled into Part B. The resultant mixture is cooled to 35° C. then the fragrance is paddle mixed into the batch.

### EXAMPLE 2

| Part | Ingredients | Wt. % |
|---|---|---|
| A | Propylene glycol | 4.0 |
| | Xanthan Gum | 0.5 |
| | Phenoxyethanol | 0.3 |
| | Demineralized water | 55.3 |
| | 5-Chlorosalicylic acid | 4.0 |
| | Sodium 5-chlorosalicylate | 1.7 |
| B | Squalane | 10.0 |
| | PPG-12/SMDI | 8.0 |
| | Hydrogenated phospholipids | 5.0 |
| | Caprylic/capric/stearic triglyceride | 2.0 |
| | Cyclopentasiloxane | 4.0 |
| | Dimethicone | 1.0 |
| | Cetearyl alcohol and ceteareth-20 | 2.0 |
| | Glyceryl stearate and PEG-100 stearate | 1.5 |
| | Steareth-2 | 0.5 |
| C | Fragrance | 0.2 |

The 5-chlorosalicylic acid and sodium 5-chlorosalicylate are slowly mixed in the demineralized water. Then the xanthan gum is slowly dispersed in the water while vigorously stirring. Mixing is continued until the gum is thoroughly dissolved. The batch is heated 75° C. then the propylene glycol is added to it followed by the phenoxyethanol.

The components of part B are combined in a separate vessel and slowly mixed while heating to 75° C. Part B is slowly milled into part A then the batch is cooled to 35° C. The fragrance is then paddle mixed into the batch.

It should be understood that the foregoing description is only illustrative of some embodiments of the present invention. Various alternatives and modifications can be devised by those skilled in the art without departing from the invention. Accordingly, the present invention is intended to embrace all such alternatives, modifications and variances that fall within the scope of the appended claims.

1. A method for treating a condition selected from the group consisting of skin requiring desquamation, nail disorders, dandruff, calluses, acne, excess sebum production, enlarged skin pore size, and blackheads, comprising contacting an area of affected skin with a composition having an effective amount of a halosalicylic acid compound of formula I, 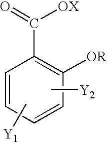
wherein X is hydrogen or a cosmetically acceptable cation; R is hydrogen, C₁-C₁₈ alkyl or C₁-C₁₈ alkyl substituted with at least one Cl, Br, F or I group; and Y₁ and Y₂ are, independently, hydrogen, Cl, Br, F, I, methyl substituted by one to three Cl, Br, F, or I groups, phenyl, or phenyl substituted by at least one substituent selected from the group consisting of C₁-C₁₈ alkyl, Cl, Br, F and I; with the proviso that at least one of Y₁ and Y₂ is Cl, Br, F or I; and a cosmetically acceptable vehicle for the halosalicylic acid compound.

2. The method as claimed in claim 1, wherein the composition contains the halosalicylic acid compound in an amount of about 0.001% to about 10% by weight, based on total weight of the composition.

3. The method as claimed in claim 1, wherein the composition contains the halosalicylic acid compound in an amount of about 0.01% to about 5% by weight, based on total weight of the composition.

4. The method as claimed in claim 1, wherein the composition contains the halosalicylic acid compound in an amount of about 0.1% to about 2.5% by weight, based on total weight of the composition.

5. The method as claimed in claim 1, wherein the composition contains the halosalicylic acid compound in an amount of about 0.5% to about 2% by weight, based on total weight of the composition.

6. The method as claimed in claim 1, wherein the compound of formula I is selected from the group consisting of 5-chlorosalicylic acid, 5-fluorosalicylic acid, 5-bromosalicylic acid, 5-iodosalicylic acid and mixtures thereof.

7. The method as claimed in claim 1, wherein the compound of formula I is 5-chlorosalicylic acid.

8. The method as claimed in claim 1, wherein the composition further contains salicylic acid.

9. The method as claimed in claim 8, wherein the salicylic acid is present in an amount of about 0.5% to about 2% by weight, based on total weight of the composition, the halosalicylic acid compound is 5-chlorosalicylic acid, and the 5-chlorosalicylic acid is present in an amount of about 0.5% to about 2% by weight, based on total weight of the composition.

10. The method as claimed in claim 1, wherein the composition further contains an RAR/RXR agonist.

11. The method as claimed in claim 1, wherein the composition further contains a 5-alpha-reductase inhibitor.

12. The method as claimed in claim 1, wherein the composition further contains an RAR/RXR agonist and a 5-alpha-reductase inhibitor.

13. The method as claimed in claim 10, wherein the RAR/RXR agonist is present in an amount of about 0.0001% to about 50% by weight, based on the total weight of the composition.

14. The method as claimed in claim 10, wherein the RAR/RXR agonist is present in an amount of about 0.01% to about 20% by weight, based on the total weight of the composition.

15. The method as claimed in claim 10, wherein the RAR/RXR agonist is present in an amount of about 0.5% to about 5% by weight, based on the total weight of the composition.

16. The method as claimed in claim 11, wherein the 5-alpha-reductase inhibitor is present in an amount of about 0.01% to about 5% by weight, based on the total weight of the composition.

17. The method as claimed in claim 11, wherein the 5-alpha-reductase inhibitor is present in an amount of about 0.1% to about 0.5% by weight, based on the total weight of the composition.

18. The method as claimed in claim 10, wherein the RAR/RXR agonist is selected from the group consisting of phytol, isophytol, phytol derivatives, isophytol derivatives, retinoids, and mixtures thereof.

19. The method as claimed in claim 10, wherein the RAR/RXR agonist is phytol, retinol or a mixture thereof

20. The method as claimed in claim 11, wherein the 5-alpha-reductase inhibitor is selected from the group consisting of oleanolic acid, saw palmetto, finasteride, and mixtures thereof.

21. The method as claimed in claim 1, wherein the composition further contains an anti-ageing active ingredient.

22. The method of claim 1, wherein the condition is skin requiring dequamation.

23. The method of claim 1, wherein the condition is enlarged skin pore size.

24. The method of claim 1, wherein the condition is excess sebum production.

25. The method of claim 1, wherein the condition is acne or blackheads.

26. A cosmetic composition comprising an effective amount of a halosalicylic acid compound of formula I, 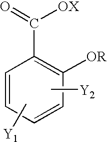
wherein X is hydrogen or a cosmetically acceptable cation; R is hydrogen, C₁-C₁₈ alkyl or C₁-C₁₈ alkyl substituted with at least one Cl, Br, F or I group; and Y₁ and Y₂ are, independently, hydrogen, Cl, Br, F, I, methyl substituted by one to three Cl, Br, F, or I groups, phenyl, or phenyl substituted by at least one substituent selected from the group consisting of C₁-C₁₈ alkyl, Cl, Br, F and I; with the proviso that at least one of Y₁ and Y₂ is Cl, Br, F or I; and a cosmetically acceptable vehicle for the halosalicylic acid compound.
